(19) United States
(12) Patent Application Publication
(10) Pub. No.: US 2017/0239313 A1
(43) Pub. Date: Aug. 24, 2017

(54) COMPOSITIONS AND METHODS COMPRISING EXTRACTS OF ZINGIBERACEAE

(71) Applicant: Biocogent, LLC, Stony Brook, NY (US)

(72) Inventors: Joseph D. Ceccoli, Farmingville, NY (US); Michael L. Ingrassia, Hauppauge, NY (US); Brian R. Costello, Port Jefferson Station, NY (US)

(21) Appl. No.: 15/519,305

(22) PCT Filed: Oct. 14, 2015
(86) PCT No.: PCT/US15/55611

§ 371 Date: Apr. 14, 2017

Related U.S. Application Data
(60) Provisional application No. 62/063,448, filed on Oct. 14, 2014.

(51) Int. Cl. A61K 36/9066; A61Q 19/08; A61Q 7/00; A61Q 19/00; A61K 9/14; A61K 8/97

(52) U.S. Cl. CPC A61K 36/9066; A61K 9/14; A61K 8/97; A61Q 7/00; A61Q 19/008; A61Q 19/08; A61K 2800/782; A61K 2800/522

(57) ABSTRACT

The present technology relates to compositions, in particular, cosmetic, topical and pharmaceutical compositions, comprising extracts of plants in the family Zingiberaceae, as well as methods in connection with such compositions, including methods of reducing sebum production, reducing the appearance of oily skin, reducing the appearance of acne, reducing the appearance of pores, reducing the appearance of wrinkles, reducing free radicals, reducing baldness, reducing or increasing hair growth and reducing or increasing androgenic stimulation, or reducing the rate of oxidation.

### BACKGROUND

The present technology relates to compositions, in particular, cosmetic and pharmaceutical compositions, comprising extracts of plants in the family Zingiberaceae, as well as methods in connection with such compositions.

### SUMMARY OF THE DISCLOSED TECHNOLOGY

In certain embodiments, the present technology is directed to a composition comprising: (a) an extract of a plant in the family Zingiberaceae; and (b) an ingredient chosen from a solvent, an emulsifier, a humectant, water, alcohol or a polymer.

In certain embodiments, the present technology is directed to a method of inhibiting an isozyme of 5α-reductase, the method comprising the step of applying a composition to a user orally or by application to the skin, wherein the composition comprises the following: (a) an extract of a plant in the family Zingiberaceae; and (b) an ingredient chosen from a solvent, an emulsifier, a humectant, water, alcohol or a polymer.

In certain embodiments, the present technology is directed to methods of reducing sebum production, reducing the appearance of oily skin, reducing the appearance of acne, reducing the appearance of pores, reducing the appearance of wrinkles, reducing free radicals, reducing baldness, reducing or increasing hair growth and reducing or increasing androgenic stimulation, or reducing the rate of oxidation; all comprising administering a composition comprising an extract of a plant in the family Zingiberaceae to a user. In various embodiments, the methods herein can involve the administration of the compositions herein to a cell, a sample of cells (including but not limited to a skin cell); to a tissue or to a user.

### BRIEF DESCRIPTION OF THE DRAWINGS

FIG. 1 shows the 5α-reductase Type I activity of exemplary compositions according to certain embodiments herein.

FIG. 2 shows the 5α-reductase Type II activity of exemplary compositions according to certain embodiments herein.

FIG. 3 shows exemplary compositions according to certain embodiments herein.

FIG. 4 shows a summary of tests conducted on the exemplary compositions

FIGS. 5A and 5B show sebum levels over time upon use of exemplary compositions according to certain embodiments herein.

FIG. 6 shows scavenging/quenching of singlet oxygen over time upon use of exemplary compositions according to certain embodiments herein.

FIG. 7 shows photographs of visible improvement of skin over time upon use of exemplary compositions according to certain embodiments herein.

FIG. 8 shows photographs of visible improvement of skin over time upon use of exemplary compositions according to certain embodiments herein.

FIGS. 9A and 9B show photographs of visible improvement of skin over time upon use of exemplary compositions according to certain embodiments herein.

FIGS. 10A, 10B and 10C show photographs of visible improvement of skin over time upon use of exemplary compositions according to certain embodiments herein.

### DETAILED DESCRIPTION

Zingiberaceae is a family of flowering plants comprising over 52 genera and over 1300 species. The family includes ginger, cardamom, turmeric and other plants that are the source of spices. Curcuma is a genus of plants in the Zingiberaceae family that are native to Asia and parts of Australia. The ground rhizome of turmeric (Curcuma longa) has a history of culinary use as both a spice and a colorant, especially in curries. Curcumin, the active component of turmeric, has been shown to have antioxidant, anti-inflammatory and anti-cancer properties. Turmeric extracts have been used for topical formulations; however, extracts of Curcuma longa have a bright yellow color that is cosmetically undesirable.

Extracts of the species Curcuma longa have been used in some cosmetic and therapeutic applications, and has been shown to have some beneficial biologic activities, such as the inhibition of 5α-reductase activity. The related species Curcuma aeruginosa has been studied in this regard as well, in connection with testosterone and androgenic compounds. See Srivilai et al., “Anti-androgenic Activity of Furanodiene Isolated From Curcuma aeruigonsa Roxb. Extract,” Naresuan University Journal, Special Issue (2011), page 33; Pumthong et al., “Curcuma aeruginosa, a Novel Botanically Derived 5α-Reductase Inhibitor in the Treatment of Male-Pattern Baldness: a Multicenter, Randomized, Double-Blind, Placebo-Controlled Study,” Journal of Dermatological Treatment 23 (2012): 385-392; Suphrom et al., “Anti-Androgenic Effect of Sesquiterpenes Isolated from the Rhizomes of Curcuma aeruginosa Roxb.,” Fitoterapia 83 (2012) 864-871. One disadvantage of Curcuma longa is the fact that its extracts impart a bright yellow color to formulated products. Such a feature would be undesirable for compositions applied to the skin.

The applicability of extracts of plants in the Zingiberaceae family in connection with cosmetic and skin formulations and methods has herein been investigated for the first time.

Androgenic stimulation is responsible for several aspects of normal skin and hair development. Increases in sebum production and body hair occur for both sexes at puberty due to elevated levels of circulating testosterone and its precursors. In skin, precursors derived from the blood are further metabolized into testosterone, which itself can be converted into the more potent form, dihydrotestosterone (DHT) by 5α-reductase enzymes. Both testosterone and DHT bind to the cytoplasmic androgen receptor (AR), a member of the steroid hormone receptor superfamily that is present in many cell types and tissues throughout the body. The resulting ligand/receptor complexes mediate their effects predominantly via transport into the nucleus where they cause changes in gene expression.

It has been shown that androgen responsive tissues such as the prostate, seminal vesicle, epididymis and skin, convert testosterone to 5α-androstan-17β-ol-3-one (5α-dihydrotestosterone, DHT). This conversion amplifies androgenic responses, perhaps because of a higher affinity of the androgen receptor for DHT than for testosterone. Such responses include, for example, induction of male pattern baldness and increased sebum production in the skin.

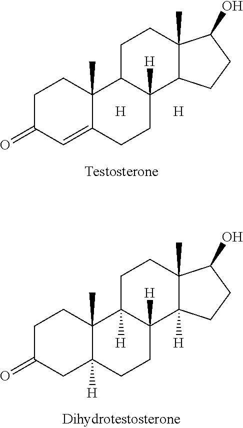

The conversion of testosterone to the more potent dihydrotestosterone (DHT) is catalyzed by the 5α-reductase enzyme. The 5α-reductase enzyme participates in 3 metabolic pathways: bile acid biosynthesis, androgen and estrogen metabolism, and prostate cancer. Specific substrates include testosterone, progesterone, androstenedione, epi-testosterone, cortisol, aldosterone, and deoxycorticosterone.

In addition to the developmental changes that are under androgenic control, several abnormal skin and hair conditions also have a strong hormonal component. Among them are androgenic alopecia (male pattern baldness and female pattern hair loss), acne vulgaris, oily skin (overproduction of sebum), and excessive/unwanted terminal (non-scalp) hair growth (hirsutism). Terminal hair growth is stimulated by AR activation while, in genetically predisposed individuals, scalp hair growth is inhibited by it, in part because it compromises nutrient absorption from the follicular blood supply.

In certain embodiments, the methods herein inhibit two or more isozymes of 5α-reductase. There are three known isozymes of 5α-reductase, with isozymes I and II relevant to the hair and skin. The type I isozyme is localized predominately in sebaceous glands while the type II isozyme is present in certain regions of terminal hair follicles.

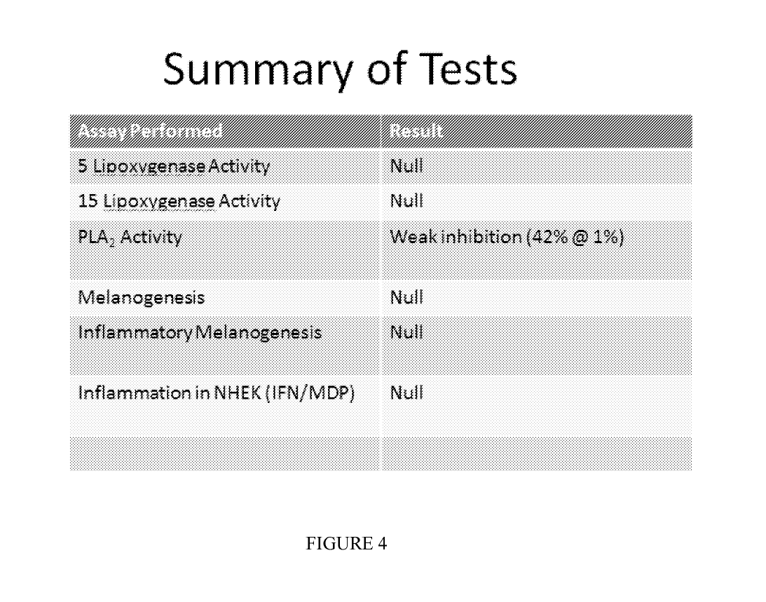

In our studies described herein, the ability of extracts of plants in the Zingiberaceae family to inhibit or otherwise alter isozymes I and II of 5α-reductase was studied. Highly substantial and statistically significant inhibition of both forms was observed. This is surprising for several reasons: among them, such activity would not be expected because (as shown in FIG. 4), certain compositions herein were found to have little or no activity when tested in connection with certain markers or mediators of inflammation (including 5 lipoxygenase, 15 lipoxygenase, PLA2, melanogenesis, inflammatory melanogenesis and inflammation in NHEK (IFN/MDP)); however, these were still unexpectedly found to inhibit or otherwise alter isozymes I and II of 5α-reductase.

For the dried extract, IC₅₀ values for both isozymes were in the range of about 0.005 to about 0.01% w/v, with complete inhibition of both isozymes occurring at about 0.05 to about 0.1% w/v. These efficacies are comparable to what can be achieved with the benchmark standard finasteride.

Thus, in certain embodiments, the present technology is directed to topical use of extracts of plants in the Zingiberaceae family to inhibit or otherwise alter 5α-reductase activity in the skin and its appendages in order to treat or achieve cosmetic benefits with respect to at least the following:

(1) undesirable skin conditions (including but not limited to “oily skin”, acne vulgaris, psoriasis, rosacea, photodermatosis, skin sensitivity, sunburn, eczema, oily scalp, oily hair and the like, which are in part characterized by abnormally high levels of sebum production;

(2) baldness (including but not limited to male pattern baldness);

(3) hirsutism, (including but not limited to hirsutism in men or women); and

(4) other skin disorders related in whole or part to elevated androgenic stimulation.

As used herein, the term “skin” includes all of the outer surfaces of the body or surfaces of the body that come into contact with agents external to the body, including the scalp, nails and mucosa. As used herein, the term “hair” includes hair on any surface of the body, including the head and elsewhere.

In certain embodiments, the compositions herein may increase or decrease hair growth, depending on the user's specific needs. For example, it has been found that increasing hair growth on the head sometimes results in decreased hair growth elsewhere on the body, and vice versa. Similarly, in certain embodiments, the compositions herein may be used to increase or decrease androgenic stimulation, again depending on the user's specific needs.

In various embodiments, the methods contemplated herein include methods of reducing or treating the incidence or appearance of any of the following: sebum production, oily skin, skin sheen, pores, blackheads, whiteheads, rough skin, cracking, pimples, acne, wrinkles, sagging skin, scarring, age spots, discoloration, free radicals, baldness (including male pattern baldness and female baldness), unwanted hair growth and androgenic stimulation. In certain embodiments, the compositions discussed herein may include one or more agents for treating any of the following: skin conditions including but not limited to acne, pimples, rosacea, wrinkles or fine lines or the appearance thereof, sunburn, sun damage, eczema, psoriasis, dandruff, allergic reactions, itchiness or inflammation.

In various embodiments, the methods contemplated herein include administering the compositions herein to a user, which includes application to a user orally (e.g., by ingesting) or through the skin (e.g., by application to the surface of the skin or mucosa, including manually rubbing a composition onto the skin or through transdermal systems), or by injection or inhalation (e.g., through the oral or nasal passages), or rectally or vaginally.

Such formulations can include but are not limited to any solid, liquid or gas formulations usable for delivery to the interior or exterior of a user's body, including but not limited to: powdered formulations, makeup entities, emulsions including microemulsions or nanoemulsions (e.g., BB creams) colloidal suspensions, oil (e.g., oil in water or water in oil emulsions), gels, ointments, lotions (e.g., moisturizers, sunscreens), foundations, solutions, aqueous compositions, aerosol compositions such as sprays, atomized compositions, mousses, mulls, hydrocolloids, sachets, pastilles, pills, chewable forms, capsules, tablets, serums, dragees, suppositories, creams and the like.

In certain embodiments, the present technology includes an extract of a plant in the family Zingiberaceae. These include, but are not limited to, the following genera of gingers: Alpinia, Globba, ginger lily (Hedychium), Kaempferia, torch-ginger Etlingera elation, Renealmia, and Zingiber.

In various embodiments, the plant is of the genus Curcuma. In certain embodiments, the species of Curcuma that are useful for the present technology include, but are not limited to, the following:

Curcuma albicoma, Curcuma albiflora, Curcuma alismatifolia (Siam tulip), Curcuma amada, Curcuma amarissima, Curcuma angustifolia, Curcuma aromatics, Curcuma attenuate, Curcuma aurantiaca, Curcuma australasica, Curcuma bakeriana, Curcuma bicolor, Curcuma bhatii, Curcuma burttii, Curcuma caesia (black turmeric), Curcuma ceratotheca, Curcuma chuanezhu, Curcuma chuanhuangjiang, Curcuma chuanyujin, Curcuma cochinchinensis, Curcuma codonantha, Curcuma coerulea, Curcuma colorata, Curcuma comosa, Curcuma coriacea, Curcuma decipiens, Curcuma ecalcarata, Curcuma euchroma, Curcuma ecomata, Curcuma data, Curcuma exigua, Curcuma ferruginea, Curcuma flaviflora, Curcuma glans, Curcuma gracillima, Curcuma grandiflora, Curcuma haritha, Curcuma harmandii, Curcuma heyneana, Curcuma inodora, Curcuma karnatakensis, Curcuma kudagensis, Curcuma kwangsiensis Curcuma lanceolata, Curcuma larsenii, Curcuma latiflora, Curcuma latifolia, Curcuma leonidii, Curcuma leucorrhiza, Curcuma loerzingii, Curcuma longa (turmeric), Curcuma longispica, Curcuma malabarica, Curcuma meraukensis, Curcuma mutabilis, Curcuma neilgherrensis, Curcuma nilamburensis, Curcuma oligantha, Curcuma ornate, Curcuma parviflora, Curcuma parvula, Curcuma peethapushpa, Curcuma petiolata, Curcuma phaeocaulis, Curcuma pierreana, Curcuma plicata, Curcuma porphyrotaenia, Curcuma prakasha, Curcuma pseudomontana, Curcuma purpurascens, Curcuma purpurea, Curcuma raktakanta, Curcuma reclinata, Curcuma rhabdota, Curcuma rhomba, Curcuma roscoeana, Curcuma rubescens, Curcuma rubrobracteata, Curcuma sattayasaii, Curcuma sichuanensis, Curcuma singularis, Curcuma sparganiifolia, Curcuma stenochila, Curcuma strobilifera, Curcuma sulcata, Curcuma sumatrana, Curcuma sylvatica, Curcuma thalakaveriensis, Curcuma thorelii, Curcuma trichosantha, Curcuma vamana, Curcuma vellanikkarensis, Curcuma wenyujin, Curcuma wenchowensis, Curcuma xanthorrhiza, Curcuma yunnanensis, Curcuma zedoaria and Curcuma zedoaroides.

In certain embodiments, a composition according to the present technology may comprise a blend of the extracts of two or more of any species of plants in the family Zingiberaceae. In one exemplary embodiment, a composition according to the present technology includes a combination of Curcuma kwangsiensis and Curcuma wenyujin. Both species are rich in sesquiterpenoids.

In certain embodiments, the present technology is directed to compositions, including but not limited to pharmaceutical or cosmetic compositions, comprising in various embodiments about 0.01 to about 100%, about 0.1 to about 99%, about 1 to about 99%, about 5 to about 95%, about 10 to about 90%, about 15 to about 75%, about 20 to about 60%; about 25 to about 50%, about 0.05 to about 5%, about 0.25 to about 10%, 0.5 to about 5%, or about 0.75 to about 2% of one or more extracts of a plant in the Zingiberaceae family. In various embodiments, the following types of compositions are further contemplated herein:

An aqueous composition comprising about 0.01 to about 60%, about 0.01 to about 40% about 0.05 to about 30% or about 0.10 to about 25% of the extract;

An emulsion comprising about 0.01 to about 60%, about 0.05 to about 55% or about 0.10 to about 50% of the extract;

A cream or lotion comprising about 0.01 to about 60%, about 0.05 to about 55% or about 0.10 to about 50% of the extract;

A paste or semisolid comprising about 40 to about 95%, about 45 to about 90%, about 50 to about 80% or about 55 to about 75% of the extract; or

A capsule, caplet, tablet, powder or other solid form comprising about 60 to about 100%, about 95 to about 100%, about 60 to about 99%, about 75 to about 98% or about 80 to about 95% of the extract.

In various embodiments, any one or more of the following additional ingredients can be used to prepare the extracts herein, or combined with the extracts contemporaneous with or after preparation of such extracts: surfactants, whether ionic, nonionic or anionic; solvents, whether organic or inorganic (including water or alcohol), humectants (e.g., propylene glycol, hexylene glycol, butylene glycol, glyceryl triacetate, neoagarobiose, sugar alcohols, polymeric polyols, urea, quillaia, aloe vera gel, MP diol, lactic acid, honey or lithium chloride), drying agents or desiccants (e.g., activated alumina, aerogel, benzophenone, bentonite clay, calcium chloride, calcium sulfate, cobalt(II) chloride, copper(II) sulfate, lithium chloride, lithium bromide, magnesium sulfate, magnesium perchlorate, potassium carbonate, silica gel, sodium chlorate, sodium chloride, sodium hydroxide, sodium sulfate or a sugar), wetting agents, dispersants, plasticizers, detergents, emulsifiers, colorants (including pigments or dyes), emollients, moisturizing agents, sunscreen agents, anti-inflammatory agents, antimicrobial agents, antibiotic agents, vitamins or minerals; as well as any other suitable cosmetic or skin care ingredients that further render such compositions suitable for application to the skin or hair.

In various embodiments, the compositions herein may further comprise botanical extracts, for example, extracts from plants (herbs, roots, flowers, fruits, stems, leaves, seeds) such as flowers, fruits, vegetables, including yeast ferment extract, padica pavonica extract, thermus thermophilis ferment extract, camelina sativa seed oil, boswellia serrata extract, olive extract, aribodopsis thaliana extract, acacia dealbata extract, acer saccharinum (sugar maple), acidophilus, acorns, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy. Further examples include, but are not limited to, tea tree oil, clove oil, Glycyrrhiza Glabra, Salix Nigra, Macrocycstis Pyrifera, Pyrus Malus, Saxifraga Sarmentosa, Vitis Vinifera, Morns Nigra, Scutellaria Baicalensis, Anthemis Nobilis, Salvia Sclarea, Rosmarinus Officianalis, Citrus Medica Limonum, Panax Ginseng, and mixtures thereof.

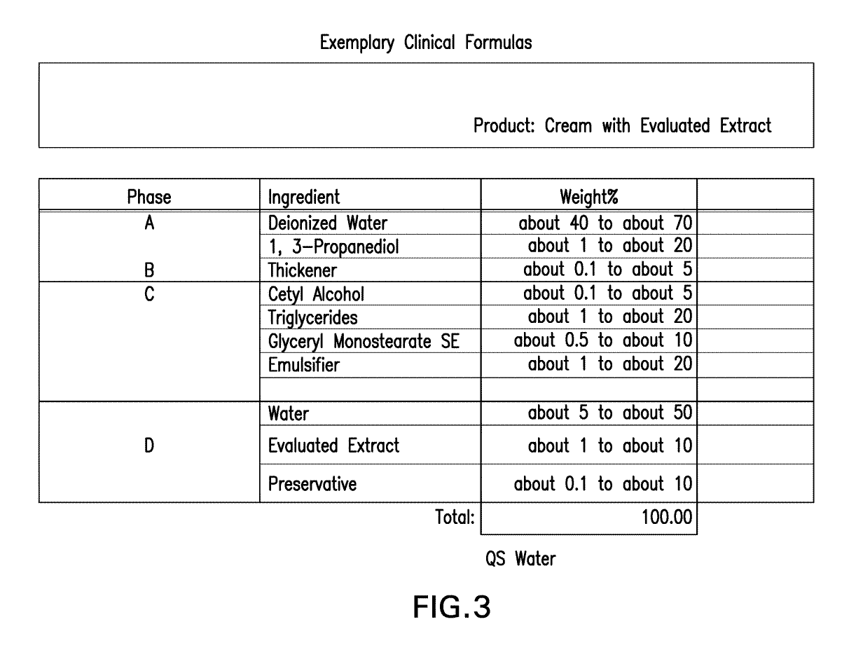

Some exemplary and non-limiting compositions herein are shown, for example, in FIG. 3, and can include, for example, deionized water, propanediol, thickener, alcohol (e.g., cetyl alcohol), triglycerides, one or more emulsifiers or one or more preservatives.

In various embodiments, the extracts herein may be encapsulated, e.g., as liposomes or the like. As used herein, the terms “encapsulated” or “encapsulate” refer to any technology that involves the confinement of one or more individual molecules within a larger molecule, or the coating of one or more particles with another material. These include, but are not limited to, liposomes, micelles, reverse micelles, vesicles; encapsulates that are opened by breaking with force, including but not limited to shell encapsulates; encapsulates that are opened by dissolving in water or solvent, including but not limited to starch encapsulates; “reservoir type” encapsulates, including but not limited to capsules, single-core, mono-core or core-shell type encapsulates; “matrix type” encapsulates; “coated matrix type” encapsulates; nanocapsules, microcapsules, microreservoir encapsulates and the like.

In certain embodiments, the extracts herein may include high energy dispersion ingredients (HEDI™ products). HEDI is a proprietary platform technology utilizing specialized, high-energy, high-shear process technology to generate unique chemical properties and the inclusion of hydrophilic and hydrophobic species together in a unified product. These include HEDI shea butter, HEDI cocoa butter, HEDI mango butter.

In certain exemplary embodiments herein, an extract of a plant in the Zingiberaceae family may be prepared through ethanol extraction to produce a slurry/liquid which is then mixed with the other excipients to prepare a composition in accordance with the technology discussed herein. In certain embodiments, the technology of the present disclosure may be in the form of encapsulates such as liposomes. In certain embodiments, the extracts herein can be prepared in powder form and then added to the other ingredients therein to produce any composition in any of the forms mentioned herein.

In various embodiments herein, the compositions herein can reduce or inhibit 5α-reductase activity by about 10% or more, about 20% or more, about 30% or more, about 40% or more, about 50% or more, about 60% or more, about 70% or more, about 80% or more; about 10 to about 95% or about 50 to about 90%.

Singlet oxygen (SO) is an electronically excited and highly reactive state of the diatomic oxygen molecule, assumed when the oxygen molecule absorbs energy. It can attack and damage biological molecules that cells rely on for proper function and survival. SO is generated from other reactive oxygen species at sites of inflammation, from peroxyl radicals such as those generated during UV-induced lipid oxidation (for example from hypochlorous acid or hydrogen peroxide), and from exposure of the skin to visible light through photosensitization reactions. Antioxidants that prevent oxidation by SO generally act in either of two ways: they can scavenge and react sacrificially with SO, thereby preventing its attack on tissue molecules; or they can quench SO b absorbing energy from it and allowing it to return to its unreactive ground state.

The compositions herein have been shown to prevent or reduce oxidation caused by SO, as discussed further herein.

Clinical formulations in accordance with certain embodiments of the present technology were subject to various testing. Experimental data are shown herein, and demonstrate desirable features including, inter alia, significant reduction of sebum production, reduction of pore size and improvement of pore and skin appearance.

### EXAMPLE 1

### 5α-Reductase Activity

5α-reductase isoenzymes were purified from normal human epidermal keratinocytes (NHEK). Extracted enzymes were incubated with testosterone and NADPH, either with or without the test compound (Evaluated Extract, about 95 to about 100% pure extract powder), and positive control (Finasteride) at 37° C. for about 30 minutes. NADPH levels at the end of the reaction were measured spectrophotometrically. Changes in NADPH levels indicate the amount of enzymatic activity in each reaction mixture.

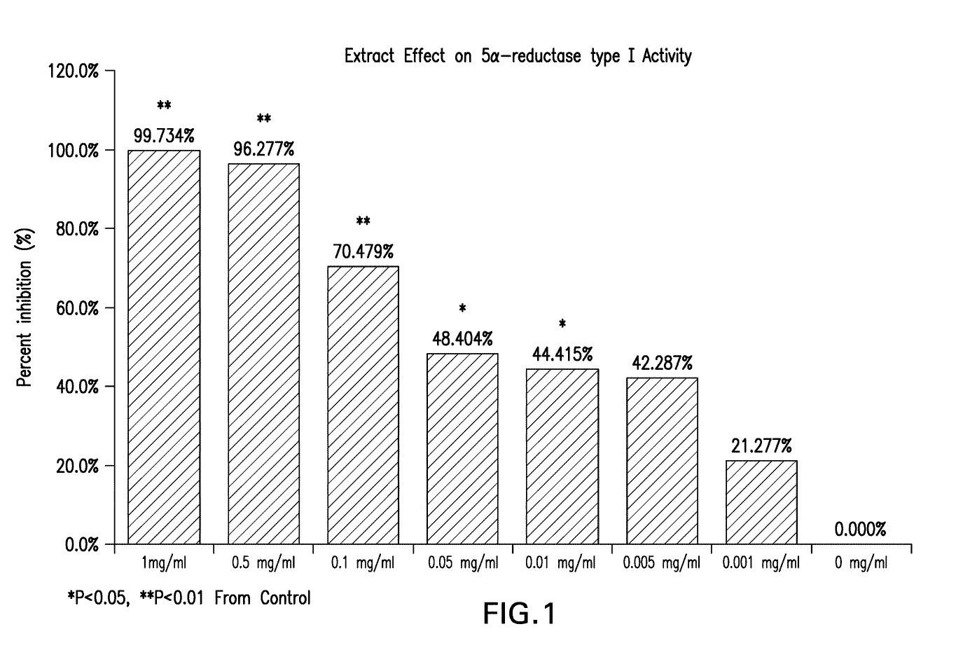

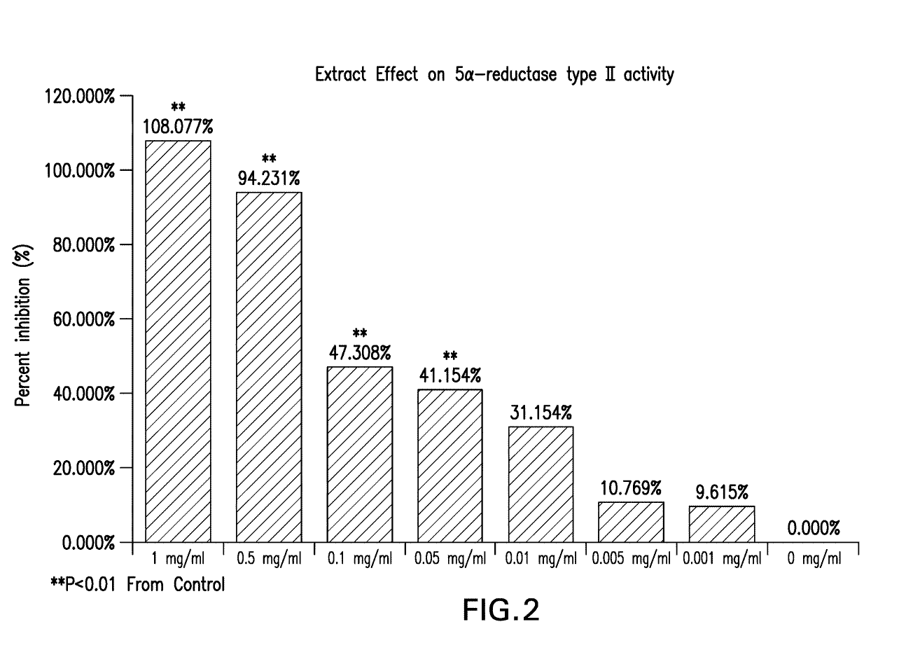

Statistical evaluation was performed using a one-way analysis of variance (ANOVA) with comparison of means by Dunnett's multiple comparison test. Results showed that the compositions herein show a notable increase in inhibition of 5α-reductase activity with increasing concentrations, as shown, e.g., in FIGS. 1 and 2.

### EXAMPLE 2

### Sebum Reduction (First Test)

Baseline sebum levels and pore size were assessed at time zero. The compositions, in the form of creams, were applied twice daily (morning & night).

Panelists were then evaluated for sebum levels using a sebumeter at 1 month.

A sebumeter SM 810 PC (Courage+Khazaka Electronic GmbH) was used to obtain measurements of skin sebum (skin surface lipids). To accomplish this, a special purpose film of the cartridge measuring head was applied for 30 seconds to the relevant skin area. The cartridge was inserted into the Sebumeter for electronic determination of film transparency variations. The LC-display of the instrument presents the result in terms of μg/cm². Duplicate measurements were obtained at the same site, and results averaged. The compositions comprised 4 different concentrations of Evaluated Extract (about 95 to about 100% pure extract powder), present in compositions in amounts of 0.1, 0.5, 1 and 2%. Results for the various concentrations are shown in Tables 1-4, respectively.

TABLE 1
Sebum Production (0.1% Evaluated Extract)

| | | | Individual % |
|---|---|---|---|
| Panelist ID | Baseline | Day 28 | Difference |
| A | 153.67 | 110.33 | −28.2 |
| B | 165.67 | 184 | 11.06 |
| C | 133.67 | 23.67 | −82.29 |
| D | 192.33 | 91.67 | −52.34 |
| E | 210 | 205.33 | −2.22 |
| Average | 171.07 | 123 | |

| | |
|---|---|
| % Difference | −28.1% |
| p | 0.132 |
| t | 1.892 |

TABLE 2
Sebum Production (0.5% Evaluated Extract)

| | | | Individual % |
|---|---|---|---|
| Panelist ID | Baseline | Day 28 | Difference |
| F | 234 | 149.67 | −36.04% |
| G | 241 | 230 | −4.56% |
| H | 188 | 227 | −20.74% |
| I | 115.67 | 38.67 | −66.57% |
| J | 168.33 | 159.67 | −5.14% |
| Average | 189.4 | 161 | |

| | |
|---|---|
| % Difference | −14.99% |
| p | 0.287 |
| t | 1.226 |

TABLE 3
Sebum Production (1% Evaluated Extract)

| | | | Individual % |
|---|---|---|---|
| Panelist ID | Baseline | Day 28 | Difference |
| K | 121.67 | 70 | −42.47% |
| L | 273.33 | 303.67 | 11.10% |
| M | 143.67 | 89 | −38.05% |
| N | 196 | 68.33 | −65.14% |
| O | 254 | 260 | 2.36% |
| Average | 197.73 | 158.2 | |

| | |
|---|---|
| % Difference | −19.99% |
| p | 0.224 |
| t | 1.439 |

TABLE 4
Sebum Production (2% Evaluated Extract)

| | | | Individual % |
|---|---|---|---|
| Panelist ID | Baseline | Day 28 | Difference |
| P | 241.33 | 200.67 | −16.85% |
| Q | 180.67 | 129 | −28.60% |
| R | 210.67 | 205.67 | −2.37% |
| S | 137.33 | 109.33 | −20.39% |
| T | 245 | 269 | 9.8% |
| Average | 203 | 182.73 | |

| | |
|---|---|
| % Difference | −9.98% |
| p | 0.208 |
| t | 1.499 |

### EXAMPLE 3

### Sebum Reduction (Second Test)

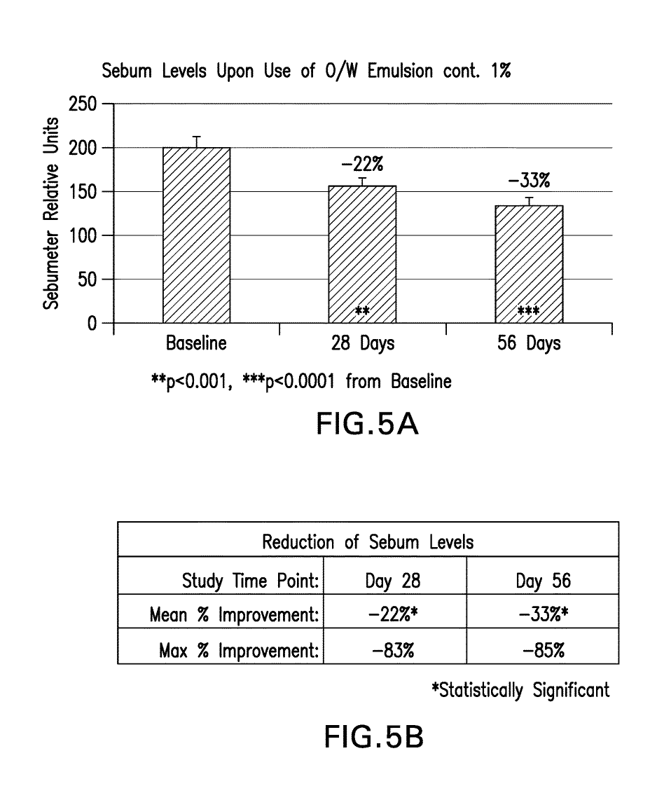

Thirty healthy adult volunteers having oily skin condition (15 males and 15 females) were recruited and tested in accordance with the procedures discussed in Example 2. Participants used a basic O/W emulsion containing an exemplary composition herein for 56 days, with twice daily application to the whole face. Compositions in accordance with certain embodiments herein were applied. Results are shown in FIGS. 5A and 5B, and indicate significant and noticeable sebum production for the test subjects.

### EXAMPLE 4

### Photographic Study

A clinical evaluation was done that included visual assessment of results based on application of compositions according to the embodiments herein.

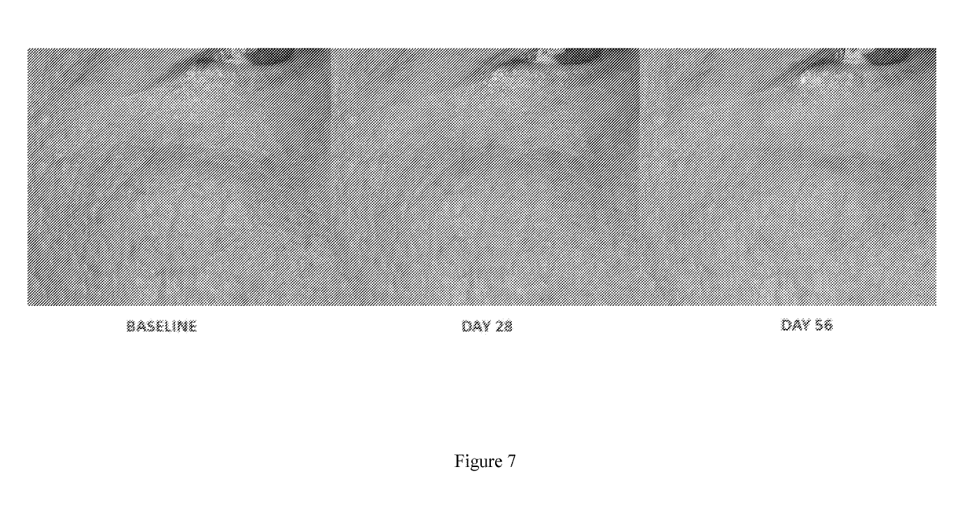

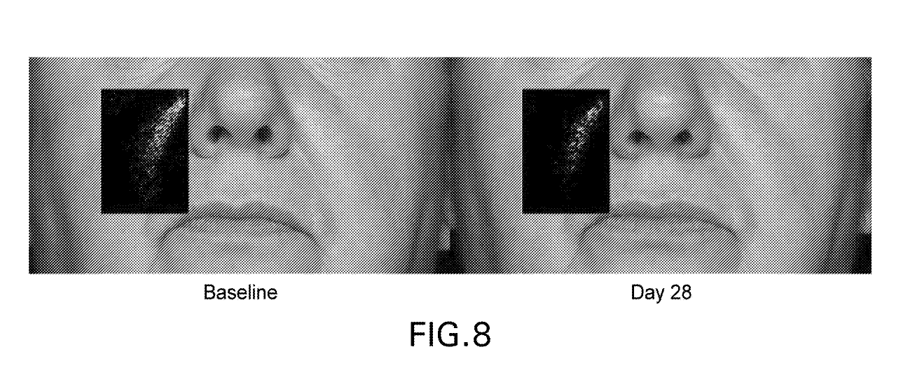

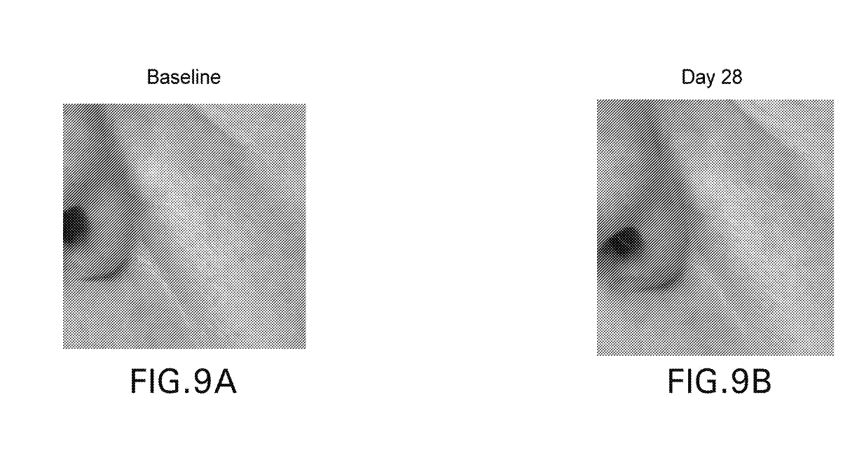

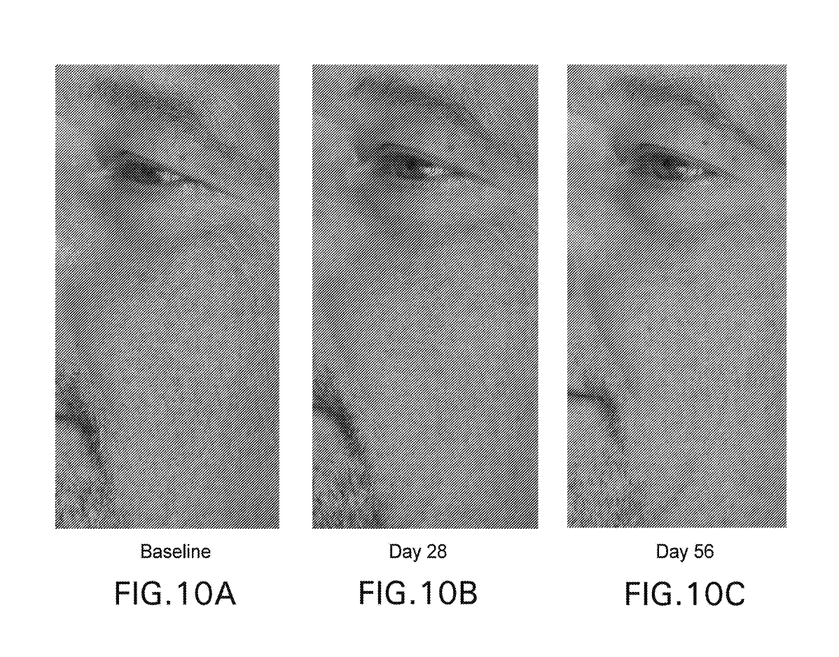

Panelists were photographed prior to application (baseline) of an exemplary composition, and again at 28 days and 56 days. High resolution photographs are shown, for example, in FIGS. 7 through 10. Visual Expert Grading was used to evaluate the condition of the skin, as shown at the bottom of each of these Figures, with 0 being the worst condition of the skin, and 10 being the best condition of the skin. Generally speaking, larger and more visible pores are undesirable, whereas smaller and less visible pores (smoother skin) is desirable.

As can be seen, for many of the panelists, visible results were obtainable regarding improvement of pore size, reduced visibility of pores, and reduction in shine. Visible results were also obtaining regarding reduction of visible redness and visible lines and wrinkles, including crows' feet. Tables 5-8 show the tabulated results of the pore size assessment.

TABLE 5
Pore Size (0.1% Evaluated Extract)

| | | | Individual % |
|---|---|---|---|
| Panelist ID | Baseline | Day 28 | Difference |
| A | 4 | 6 | 33.33% |
| B | 7 | 8 | 33.33% |
| C | 4 | 5 | 16.67% |
| D | 3 | 4 | 14.29% |
| E | 2 | 3 | 12.5% |
| Average | 4 | 5.2 | |

| | |
|---|---|
| % Difference | 20% |
| p | 0.004 |
| t | 6 |

TABLE 6
Pore Size (0.5% Evaluated Extract)

| | | | Individual % |
|---|---|---|---|
| Panelist ID | Baseline | Day 28 | Difference |
| F | 3 | 5 | 28.57 |
| G | 3 | 5 | 28.57 |
| H | 5 | 6 | 20 |
| I | 7 | 8 | 33.33 |
| J | 3 | 3 | 0 |
| Average | 4.2 | 5.4 | |

| | |
|---|---|
| % Difference | 20.69% |
| p | 0.033 |
| t | 3.207 |

TABLE 7
Pore Size (1% Evaluated Extract)

| | | | Individual % |
|---|---|---|---|
| Panelist ID | Baseline | Day 28 | Difference |
| K | 4 | 6 | 33.33% |
| L | 3 | 5 | 28.57% |
| M | 4 | 5 | 16.67% |
| N | 2 | 5 | 37.5% |
| O | 3 | 4 | 14.29% |
| Average | 43.2 | 5 | |

| | |
|---|---|
| % Difference | 26.47% |
| p | 0.009 |
| t | 4.811 |

TABLE 8
Pore Size (2% Evaluated Extract)

| | | | Individual % |
|---|---|---|---|
| Panelist ID | Baseline | Day 28 | Difference |
| P | 3 | 4 | 14.29% |
| Q | 4 | 5 | 16.67% |
| R | 1 | 2 | 11.11% |
| S | 5 | 5 | 0 |
| T | 3 | 5 | 28.57% |
| Average | 3.2 | 4.2 | |

| | |
|---|---|
| % Difference | 14.71% |
| p | 0.034 |
| t | 3.162 |

### EXAMPLE 5

### Scavenging/Quenching of Singlet Oxygen

Exemplary compositions as described herein were tested for their ability to prevent oxidation caused by SO. Reaction mixtures containing a photosensitizing dye were exposed to visible light to generate SO. Oxidation of iodide by SO was measured spectrophotometrically. The test measured oxidation of Iodide (I⁻) to form triiodide (I₃⁻), which absorbs light at a wavelength of 355 nanometers. The reaction mixture contained a photosensitizing dye (Rose Bengal) for generation of SO upon exposure to visible light. The SO then oxidized iodide ions. The rate of production of triiodide under visible light illumination was determined by measuring the increase in optical density of the reaction mixture at 355 nanometers (that is, the oxidation rate). Any test substance that is an effective antioxidant should decrease this rate. The percentage decrease of the rate of oxidation in the rate of a test sample is presented as its “% antioxidant activity.” The EC50 value is the concentration of a test sample that decreases the oxidation rate by 50%; the EC90 value is the concentration of a test sample that decreases the oxidation rate by 90%.

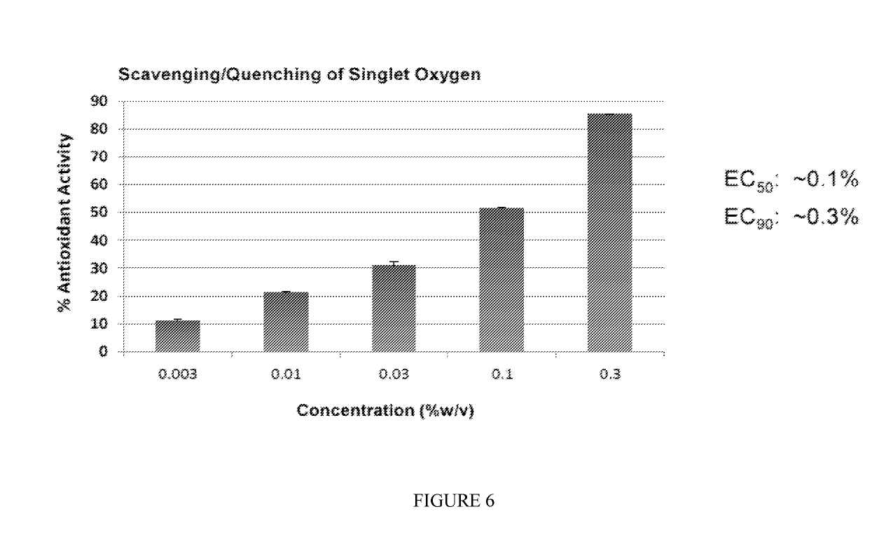

Results are shown in FIG. 6. As shown therein, the exemplary compositions herein showed good antioxidant activity, preventing SO-mediated oxidation. The EC50 and EC90 values are shown to be about 0.1% and 0.3%, respectively. In various embodiments herein, the range of these values can be about 0.05 to about 1% about 0.1 to about 0.75%, about 0.05 to about 5%, about 0.1 to about 3% or about 0.1 to about 1% or about 0.1 to about 0.3%.

Other results shown included the following: improvement in skin texture of about 10% or more, about 25% or more or about 30% or more; reduction in pore size of about 10% or more, about 25% or more, or about 45% or more; reduction in visible shine of about 10% or more, about 25% or more or about 30% or more. Further, in various embodiments, a reduction of oil present was found as follows: after 1 month, about 10% or more or about 15% or more; after 2 months, about 20% or more or about 30% or more.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

What is claimed:

1. A composition comprising:
(a) an extract of a plant in the family Zingiberaceae; and
(b) an ingredient chosen from a solvent, an emulsifier, a humectant, water, alcohol or a polymer.

2. The composition of claim 1, wherein the extract is an extract of a plant of genus Curcuma.

3. The composition of claim 2, wherein the extract is in a solid, liquid or encapsulated form.

4. The composition of claim 1, wherein the composition is in the form of any of the following: a powdered formulation, makeup entity, makeup emulsion, colloidal suspension, oil, gel, ointment, lotion, solution, aqueous composition, aerosol composition, atomized composition, mousse, mull, hydrocolloid, sachet, pastille, pill, chewable form, capsule, tablet, serum, dragee, suppository or cream.

5. The composition of claim 1, comprising about 0.05 to about 5% an extract of a plant in the family Zingiberaceae, wherein the extract is in powder form.

6. A method of inhibiting an isozyme of 5α-reductase, the method comprising the step of applying a composition to a user orally or by application to the skin, wherein the composition comprises the following:
(a) an extract of a plant in the family Zingiberaceae; and
(b) an ingredient chosen from a solvent, an emulsifier, a humectant, water, alcohol or a polymer.

7. The method of claim 6, wherein the extract is an extract of a plant of genus Curcuma.

8. A method of reducing sebum production, the method comprising administering a composition according to claim 1 to a user.

9. A method of reducing the appearance of oily skin, acne, wrinkles, sagging skin, scarring, pores, age spots or discoloration, the method comprising administering a composition according to claim 1 to a user.

10. A method of reducing or increasing hair growth, the method comprising administering a composition according to claim 1 to a user.

11. A method of reducing or increasing androgenic stimulation in a cell, the method comprising administering a composition according to claim 1 to a user.

12. A method of reducing free radicals in a cell, the method comprising administering a composition according to claim 1 to a user.

13. A method of decreasing the rate of oxidation in a cell, the method comprising administering a composition according to claim 1 to the cell.

14. The method of claim 13, wherein the rate of oxidation is decreased by about 50% or more.
